(19) United States
(12) Patent Application Publication
(10) Pub. No.: US 2025/0195785 A1
(43) Pub. Date: Jun. 19, 2025

(54) SUB-ASSEMBLIES FOR MEDICAMENT DELIVERY DEVICES AND CORRESPONDING METHODS OF ASSEMBLY

(71) Applicant: SHL Medical AG, Zug (CH)

(72) Inventors: Oscar Alexandersson, Nacka Strand (SE); Carrie Zhong, Nacka Strand (SE)

(21) Appl. No.: 18/846,372

(22) PCT Filed: Mar. 8, 2023
(86) PCT No.: PCT/EP2023/055851

§ 371 Date: Sep. 12, 2024

(30) Foreign Application Priority Data
Mar. 22, 2022 (EP) 22163564.2

(51) Int. Cl. A61M 5/315

(52) U.S. Cl. CPC A61M 5/31586; A61M 2207/00

(57) ABSTRACT

The present disclosure provides a sub-assembly for a medicament delivery device, the sub-assembly comprising a housing and a rear cap arranged coaxially in the housing. The housing is tubular and extends from a proximal end to a distal end along a longitudinal axis in an axial direction, and the housing includes an inner surface and an outer surface. The inner surface of the housing includes a first surface facing in the distal direction and a second surface facing in the distal direction. The second surface is closer to the distal end of the housing than the first surface. The rear cap extends from a proximal end to a distal end along the longitudinal axis. The rear cap includes a flexible arm, the flexible arm comprising a first surface facing in the proximal direction, and the rear cap comprises a second surface facing in the proximal direction.

### CROSS-REFERENCE TO RELATED APPLICATIONS

The present application is a U.S. National Phase Application pursuant to 35 U.S.C. § 371 of International Application No. PCT/EP2023/055851 filed Mar. 8, 2023, which claims priority to European Patent Application No. 22163564.2 filed Mar. 22, 2022. The entire disclosure contents of these applications are herewith incorporated by reference into the present application.

### TECHNICAL FIELD

The invention concerns sub-assemblies for medicament delivery devices, and particularly sub-assemblies comprising a housing and a rear cap.

### BACKGROUND

Patent applications WO2016/169748 and WO2021/204499 show medicament delivery devices comprising a rear cap and a housing. The applicant has appreciated that further improvements can be made to the design of the rear cap and the housing.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a sub-assembly for a medicament delivery device, the sub-assembly comprising a housing and a rear cap arranged coaxially in the housing, wherein the housing is tubular and extends from a proximal end to a distal end along a longitudinal axis in an axial direction, wherein the housing comprises an inner surface and an outer surface, the inner surface of the housing comprising a first surface facing in the distal direction and a second surface facing in the distal direction, wherein the second surface is closer to the distal end of the housing than the first surface, wherein the rear cap extends from a proximal end to a distal end along the longitudinal axis, the rear cap comprising a flexible arm, the flexible arm comprising a first surface facing in the proximal direction, wherein the rear cap comprises a second surface facing in the proximal direction, wherein the second surface of the rear cap is closer to the distal end of the rear cap than the first surface of the rear cap, wherein the distance in the axial direction between the first surface of the housing and the second surface of the housing is smaller than the distance in the axial direction between the first surface of the rear cap and the second surface of the rear cap, so that when the rear cap is inserted into the distal end of the housing during assembly, the first surface of the rear cap engages the first surface of the housing and thereby flexes the flexible arm before the second surface of the rear cap engages the second surface of the housing. This structure enables a transportation lock to be maintained even after the rear cap has started to be inserted into the housing, as the flexible arms can maintain a transport lock for longer than in the existing designs. This structure enables designs in which the period when a powerpack is unlocked during assembly is reduced or eliminated.

Optionally, the sub-assembly comprises a powerpack, wherein the powerpack comprises the rear cap, and wherein the sub-assembly is configured so that flexing of the flexible arm of the rear cap during insertion of the rear cap into the housing unlocks the powerpack.

Optionally, the sub-assembly is configured so that the flexible arm flexes perpendicular to the longitudinal axis when the first surface of the rear cap engages the first surface of the housing.

Optionally, the powerpack comprises a rotator, and wherein flexing the flexible arm disengages a rotational lock between the rear cap and the rotator.

Optionally, the sub-assembly comprises a medicament delivery member guard, wherein the rotator comprises a groove, and wherein the medicament delivery member guard comprises a protrusion that is configured to rotationally lock the rotator by engaging the groove.

Optionally, the inner surface of the housing comprises a first portion at a first distance from the longitudinal axis, a second portion at a second distance from the longitudinal axis, and an intermediate portion that extends from the first portion to the second portion, the intermediate portion comprising the first surface. Optionally, the second surface of the housing is at the distal end of the second portion.

Optionally, when in an unflexed state, the flexible arm is closer to the longitudinal axis than the second portion of the inner surface of the housing.

Optionally, at least one of the first surface of the rear cap and the first surface of the housing are angled relative to the longitudinal axis. Optionally, the first surface of the housing is angled towards the longitudinal axis. Optionally, the second surface of the housing and/or the second surface of the rear cap extend perpendicular to the longitudinal axis. Optionally, the second surface of the housing is at the distal end of the housing.

An aspect concerns a medicament delivery device comprising the sub-assembly of any previous claim. Optionally, the medicament delivery device is an autoinjector.

An aspect concerns a kit of parts comprising any housing as described above and any rear cap as described above.

An aspect concerns a method of assembling a sub-assembly of a medicament delivery device, the method comprising the following steps in the following order: providing any housing as described above and any rear cap as described above; inserting the rear cap partially into the distal end of the housing; and pushing the rear cap in the proximal direction relative to the housing so that the first surface of the rear cap engages the first surface of the housing and thereby flexes the flexible arm before the second surface of the rear cap engages the second surface of the housing. Optionally, the sub-assembly comprises a medicament delivery member guard and a powerpack, the powerpack comprising the rear cap and a rotator, and wherein a rotational lock between the rear cap and the rotator is only released after a rotational lock between the rotator and the medicament delivery member guard has been achieved.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a partially see-through side view of a distal part of an autoinjector during assembly.

FIG. 2 shows a cross-section side view of the autoinjector of FIG. 1 at the same point in assembly as in FIG. 1.

FIG. 3 shows a perspective view of part of the autoinjector of FIG. 1.

FIG. 4 shows a cross-section side view of the autoinjector of FIG. 1 during assembly.

FIG. 5 shows a cross-section side view of the autoinjector of FIG. 1 after assembly.

FIG. 6 shows a perspective view of the components of a powerpack, a needle guard and a housing of the autoinjector of FIG. 1.

### DETAILED DESCRIPTION

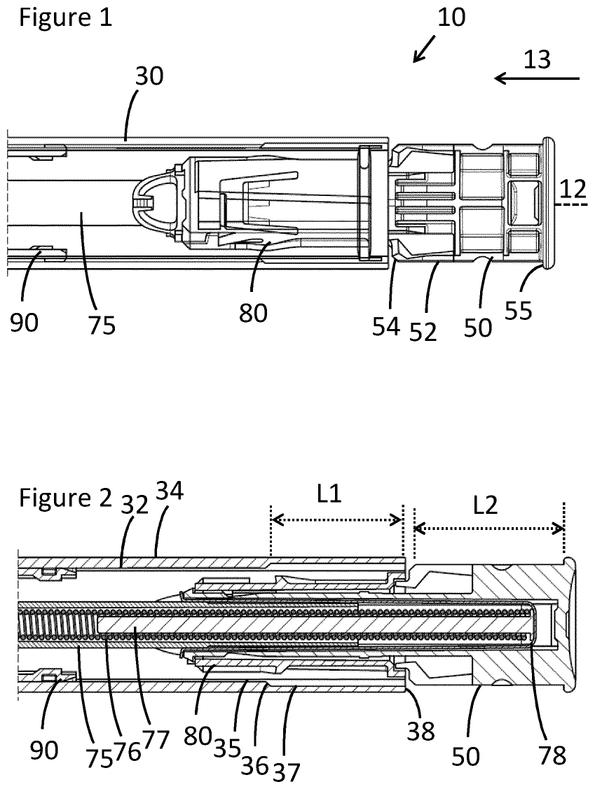

FIG. 1 shows part of an autoinjector 10 during assembly. The autoinjector extends in an axial direction 13 along an axis 12. The autoinjector comprises a sub-assembly comprising a housing 30 and a rear cap 50. The rear cap 50 is arranged coaxially in the housing. The housing 30 is tubular and extends from a proximal end to a distal end along a longitudinal axis in an axial direction, with the housing 30 comprising an outer surface 32 and an inner surface 34. In this example, the inner surface 34 comprises a first portion 35 at a first distance from the longitudinal axis, a second portion 37 at a second distance from the longitudinal axis, and an intermediate portion 36 (first surface of the housing) that extends from the first portion to the second portion. The second portion 37 is closer to the distal end of the tube than the first portion 35. The intermediate portion 36 comprises a first surface of the housing, and the housing also comprises a second surface 38. Both the first surface of the housing and the second surface of the housing face towards the distal end of the tube.

The rear cap 50 extends from a proximal end to a distal end, with the rear cap comprising a flexible arm 52. The flexible arm 52 comprises a first surface 54 facing towards the proximal end of the rear cap 50. The rear cap 50 comprises a second surface 55 facing towards the proximal end of the rear cap. The second surface 55 of the rear cap 50 is closer to the distal end of the rear cap than the first surface 54 of the rear cap.

The distance L1 in the axial direction between the first surface of the housing and the second surface of the housing is smaller than the distance L2 in the axial direction between the first surface of the rear cap and the second surface of the rear cap (see FIG. 2), so that when the rear cap is inserted into the distal end of the housing during assembly, the first surface of the rear cap engages the first surface of the housing and thereby flexes the flexible arm before the second surface of the rear cap engages the second surface of the housing, as described in more detail below.

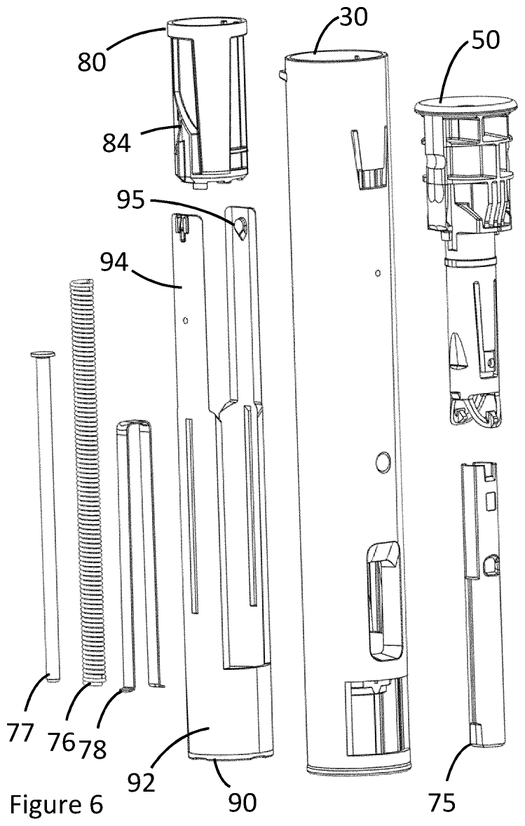

During assembly, the rear cap 50 is inserted into the distal end of the housing 30 to a distal position relative to the housing 30, as shown in FIG. 4. In the distal position as shown in FIG. 4, the first surface of the rear cap and the first surface of the housing are in contact with one another, and the second surface of the rear cap and the second surface of the housing are spaced apart. The rear cap 50 is subsequently pushed further in the proximal direction relative to the housing, resulting in the proximal position shown in FIG. 5. In the proximal position as shown in FIG. 5, the second surface of the rear cap and the second surface of the housing are in contact with one another. The first surface of the rear cap and the first surface of the housing are spaced apart from one another.

The rear cap 50 is typically part of a powerpack of the autoinjector, and the housing is typically part of a front assembly of the autoinjector. For context, various other parts of the example powerpack are shown in the Figures, including a plunger rod 75, a medicament delivery spring 76 for pushing the plunger rod in the proximal direction to expel a medicament, a guide rod 77 inside the spring 76, and a U-bracket 78 for providing feedback (such as an end click) to indicate progress of medicament delivery. These features are all optional, and could be altered or removed depending on the powerpack design. In this example, a spring 76 is used to provide the power needed to expel a medicament, but another power source such as a battery or compressed gas could alternatively be used. Similarly, a medicament delivery member guard (needle guard) 90 (which is generally part of the front assembly) and a rotator 80 (which is generally part of the powerpack) are shown, which can be used for activation of the autoinjector, but these are also optional and could be replaced with an alternative mechanism such as a button for activation of the power source used for medicament delivery.

The structure of the housing and the rear cap allow for the provision of a transport lock (for example a rotational lock). The exact nature of the transport lock can vary, but one example is shown in the figures and will be described briefly; for a more detailed description of the structure of an example powerpack, reference is made to WO2011/123024, which is incorporated herein by reference. In the example in the present application, the medicament delivery spring 76 is stopped from pushing the plunger rod 75 in the proximal direction (and thereby expelling medicament) by a second arm of the rear cap 50; the second arm is blocked from releasing the plunger rod 75 by the rotator 80. Once the rotator 80 rotates, the second arm is no longer blocked by the rotator 80, and the second arm can move to release the plunger rod. The rotator 80 can be rotated by axial movement of the needle guard 90. To avoid premature unlocking of the powerpack, it is beneficial that the rotator cannot be accidentally rotated to an unlocked position prematurely. As a result, it is beneficial to lock the rotator rotationally. This can allow, for example, the powerpack to be transported in a locked state, which can reduce the risk of accidental activation during assembly.

For context, FIG. 6 shows an example of the components of a powerpack, along with a medicament delivery member shield and a housing, for a medicament delivery device (in this case an autoinjector, although the concepts described herein could also be used in other medicament delivery devices) including the sub-assemblies described herein. The housing 30, the rear cap 50, the medicament delivery spring 76, the guide rod 77, the U-bracket 78 and the rotator 80, and the needle guard 90 are visible. The needle guard 90 comprises a base 92 and two arms 94, with each arm comprising an inwardly facing protrusion 95, which can engage a labyrinth 84 on the rotator 80. In the depicted example, the housing 30 is an outer housing of the medicament delivery device, although the housing could alternatively be inside the medicament delivery device rather than providing the outer housing. The particular shape of the rotator 80 (and therefore also of the recess 82 and the labyrinth 84) could be varied, for example depending on the shape of the flexible arm 52 and the shape of the needle guard 90. Similarly, the needle guard 90 (and therefore also the base 92, the arms 94 and the inwardly facing protrusions 95) could be varied, for example depending on the shape of the housing 30 and the rotator 80. The needle guard 90 could have one, three or more arms 94 rather than the depicted two arms.

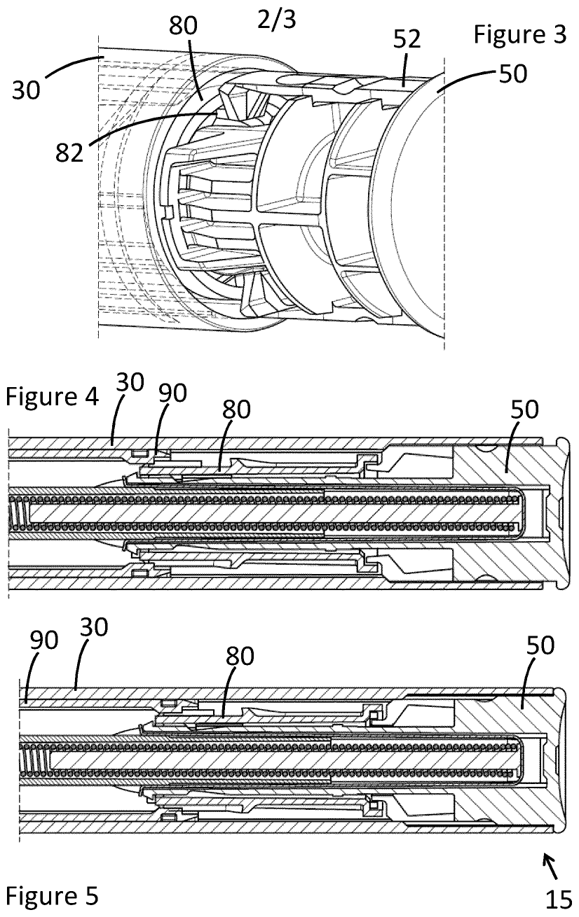

In the depicted example, the powerpack lock is provided by a combination of a recess 82 in the rotator, into which the flexible arm 52 extends see FIG. 3), and the flexible arm 52. Specifically, a portion of the flexible arm extends into the recess 82 of the rotator (in the depicted example it is a protrusion of the flexible arm that extends in the axial direction at the proximal end of the flexible arm that extends into the recess 82, although this could alternatively be a different part of the flexible arm, and could be spaced apart from the proximal end of the flexible arm). When the powerpack, including the rear cap and the rotator, is inserted into the housing (i.e. moving the components from the position in FIG. 1 to the position in FIG. 4), the powerpack lock remains in place. Only once the flexible arms 52 are pushed inwards, as shown in FIG. 5, is the powerpack lock (the engagement between the flexible arm 52 and the recess 82) unlocked. By this point, the needle guard 90 and the rotator 80 are engaged (specifically the labyrinth 84 and the inwardly facing protrusion 95), so that by the time the powerpack lock is released, a part of an arm 94 (specifically the inwardly facing protrusion 95) of the needle guard 90 engages a groove on the rotator (the groove being part of the labyrinth 84), restricting the rotator from rotation relative to the needle guard, and therefore also relative to the housing, as the needle guard is rotationally locked relative to the housing. This can reduce (and in this case eliminate) the time during this assembly step where the powerpack is unlocked. In this design, there is never a point at which the rotator is free to move relative to the rear cap, as it is either restricted from rotation by the powerpack lock (flexible arm 52 and recess 82) or by the needle guard 90.

As mentioned above, this particular powerpack structure is not essential, and could be varied. For example, a button mechanism could be provided as the activation mechanism for a medicament delivery device as well as or instead of the combination of a needle guard and a rotator as described above, or a different power source (e.g. compressed gas or a battery) could be provided. In each of these cases, the combination of the housing and the rear cap as described herein can be used to limit or entirely eliminate time spent during assembly in which the powerpack can be activated, by providing an arm 52 which is only moved aside once the rear cap is at least partly inside the housing, as this unlocks the powerpack later in the assembly process.

In the depicted example, the flexible arm is pushed towards the axis by the interaction between the first surface of the rear cap and the first surface of the housing. Alternatively, the flexible arm could be pushed in a different direction, for example circumferentially or axially. In general, the flexible arm just needs to be moved relative to another part of the rear cap (and therefore relative to another part of the powerpack, such as the rotator in the depicted example) to remove the powerpack lock, with the structure of the lock modified accordingly. In the depicted example, the lock is therefore between the flexible arm of the rear cap and the rotator, but the lock could alternatively be between the rear cap and another component of a powerpack, particularly in powerpacks that do not include a rotator.

In the depicted example, the flexible arm is closer to the axis than the second portion of the inner surface of the housing when in the unflexed state. This is not essential (as the second surface 38 could push the flexible arm 52 partly inwards to arrive at the position in FIG. 4), but can minimise the force required to place the rear cap in the position shown in FIG. 4 (where the first surfaces of the flexible arm and the housing are in contact).

In the depicted example, the housing is cylindrical with a circular cross-section, although different shapes could be used. In the depicted example, a first portion 35, an intermediate portion 36 and a second portion 37 are described as being part of the inner surface 34 of the housing 30. In this example, the first portion 35 and the second portion 37 are cylindrical, and the intermediate portion 36 is frustoconical. This can be beneficial, as provision of rotational symmetry means that the rotational alignment of the powerpack (specifically the rear cap) and the housing during assembly is not needed to disengage the powerpack lock. Alternatively, the first portion 35, the intermediate portion 36 and/or the second portion 37 could extend only part of the way around the axis. Multiple first portions 35, intermediate portions 36 and/or second portions 37 could be provided—for example two of each, to align with the two flexible arms 52 in the depicted example. It is also optional that the first portion 35 and the second portion 37 are cylindrical, and different shaped surfaces could instead be used, although cylindrical surfaces may have the advantage of efficiently minimising volume usage.

In general, the intermediate portion 36 provides a surface facing in the distal direction. This can deflect the flexible arm during assembly. In practice, the intermediate portion 36 is angled towards the surface, with the angle being the angle of the intermediate portion 36 relative to the axis 12 when viewed in a longitudinal cross-section (as shown in FIG. 2, for example). The angle could be between 15 and 90 degrees for example, or more particularly between 30 and 75 degrees. If an angle of 90 degrees is used (i.e. perpendicular to the axis 12), the corresponding surface of the flexible arm would need to be angled relative to the axis 12. With an angled surface (i.e. not 90 degrees), the surface of the intermediate portion 36 faces both in the distal direction and towards the axis. In general, the surface of the intermediate portion 36 is not parallel to the axis 12. Optionally, the angle of the intermediate portion 36 and the angle of the surface 54 of the flexible arm 52 are the same.

In general, the flexible arm comprises a surface 54 facing in the proximal direction. The surface of the flexible arm engages the intermediate portion 36 during assembly. The surface of the flexible arm can be perpendicular to the axis 12 or at an angle relative to the axis. The angle could be between 15 and 90 degrees for example, or more particularly between 30 and 75 degrees. If an angle of 90 degrees is used (i.e. perpendicular to the axis 12), the corresponding surface of the intermediate portion 36 would need to be angled relative to the axis 12. With an angled surface (i.e. not 90 degrees), the surface of the flexible arm faces both in the distal direction and away from the axis. In general, the surface of the flexible arm 52 is not parallel to the axis 12. The rear cap in the depicted example has two flexible arms; alternatively, one, three or more flexible arms 52 could be provided.

In examples where the flexible arm flexes in a direction other than a radial direction relative to the axis as in the depicted example (for example in a circumferential direction relative to the axis), the intermediate portion 36 and the surface 54 of the flexible arm 52 could be angled accordingly, and would not necessarily face towards/away from the axis.

In the depicted example, the second surface 38 of the housing 30 and the second surface 55 of the rear cap 50 are both perpendicular to the axis. However, alternative angles for the second surface 38 of the housing 30 and the second surface 55 of the rear cap 50 are also possible in the same way as for the intermediate portion 36 and the first surface 54, and additionally the second surface of the housing could face away from the axis 12 (and correspondingly the second surface of the rear cap could face towards the axis 12). The second surface 38 of the housing 30 and the second surface 55 of the rear cap 50 are optional, but can be useful to establish a fixed axial position for the housing relative to the rear cap in a completed medicament delivery device. The second surface 38 of the housing is at the distal end of the housing in the depicted example, but could alternatively be spaced apart in the axial direction from the distal end of the housing.

The delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-15. (canceled)

16. A sub-assembly for a medicament delivery device, the sub-assembly comprising a housing and a rear cap arranged coaxially in the housing,
wherein the housing is tubular and extends from a proximal end to a distal end along a longitudinal axis in an axial direction,
wherein the housing comprises an inner surface and an outer surface, the inner surface of the housing comprising a first surface facing in the distal direction and a second surface facing in the distal direction,
wherein the second surface is closer to the distal end of the housing than the first surface,
wherein the rear cap extends from a proximal end to a distal end along the longitudinal axis, the rear cap comprising a flexible arm, the flexible arm comprising a first surface facing in the proximal direction,
wherein the rear cap comprises a second surface facing in the proximal direction, wherein the second surface of the rear cap is closer to the distal end of the rear cap than the first surface of the rear cap,
wherein the distance in the axial direction between the first surface of the housing and the second surface of the housing is smaller than the distance in the axial direction between the first surface of the rear cap and the second surface of the rear cap, so that when the rear cap is inserted into the distal end of the housing during assembly, the first surface of the rear cap engages the first surface of the housing and thereby flexes the flexible arm before the second surface of the rear cap engages the second surface of the housing.

17. The sub-assembly of claim 16, wherein the sub-assembly comprises a powerpack, wherein the powerpack comprises the rear cap, and
wherein the sub-assembly is configured so that flexing of the flexible arm of the rear cap during insertion of the rear cap into the housing unlocks the powerpack.

18. The sub-assembly of claim 16, wherein the sub-assembly is configured so that the flexible arm flexes perpendicular to the longitudinal axis when the first surface of the rear cap engages the first surface of the housing.

19. The sub-assembly of claim 17, wherein the powerpack comprises a rotator, and wherein flexing the flexible arm disengages a rotational lock between the rear cap and the rotator.

20. The sub-assembly of claim 19, wherein the sub-assembly comprises a medicament delivery member guard, wherein the rotator comprises a groove, and wherein the medicament delivery member guard comprises a protrusion that is configured to rotationally lock the rotator by engaging the groove.

21. The sub-assembly of claim 16, wherein the inner surface of the housing comprises a first portion at a first distance from the longitudinal axis, a second portion at a second distance from the longitudinal axis, and an intermediate portion that extends from the first portion to the second portion, the intermediate portion comprising the first surface, and wherein the second surface of the housing is at the distal end of the second portion.

22. The sub-assembly of claim 21, wherein, when in an unflexed state, the flexible arm is closer to the longitudinal axis than the second portion of the inner surface of the housing.

23. The sub-assembly of claim 16, wherein at least one of the first surface of the rear cap and the first surface of the housing are angled relative to the longitudinal axis.

24. The sub-assembly of claim 16, wherein the first surface of the housing is angled towards the longitudinal axis.

25. The sub-assembly of claim 16, wherein the second surface of the housing and/or the second surface of the rear cap extend perpendicular to the longitudinal axis.

26. The sub-assembly of claim 16, wherein the second surface of the housing is at the distal end of the housing.

27. A medicament delivery device comprising the sub-assembly of claim 16.

28. A kit of comprising the housing as described in claim 16 and the rear cap as described in claim 16.

29. A method of assembling a sub-assembly of a medicament delivery device, the method comprising the following steps in the following order:
providing a housing as described in claim 16 and a rear cap as described in claim 16;
inserting the rear cap partially into the distal end of the housing; and
pushing the rear cap in the proximal direction relative to the housing so that the first surface of the rear cap engages the first surface of the housing and thereby flexes the flexible arm before the second surface of the rear cap engages the second surface of the housing.

30. The method of claim 29, wherein the sub-assembly comprises a medicament delivery member guard and a powerpack, the powerpack comprising the rear cap and a rotator, and wherein a rotational lock between the rear cap and the rotator is only released after a rotational lock between the rotator and the medicament delivery member guard has been achieved.
